(19) United States
(12) Patent Application Publication
(10) Pub. No.: US 2010/0228116 A1
(43) Pub. Date: Sep. 9, 2010

(54) SYSTEM AND METHOD OF OPTIMIZING A HETEROGENEOUS RADIATION DOSE TO BE DELIVERED TO A PATIENT

(76) Inventors: Weiguo Lu, Madison, WI (US); Kenneth J. Ruchala, Madison, WI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/717,086

(22) Filed: Mar. 3, 2010

Related U.S. Application Data
(60) Provisional application No. 61/157,062, filed on Mar. 3, 2009.

(51) Int. Cl. A61N 5/00; A61B 5/055; G06G 7/60; G06G 7/62

(52) U.S. Cl. 600/411; 250/492.1; 600/1; 703/11; 703/6

(57) ABSTRACT

A radiation therapy treatment system and method of optimizing a heterogeneous dose to be delivered to a patient. The system includes a computer processor and a software program stored in a computer readable medium accessible by the computer processor. The software program is operable to receive a prescribed heterogeneous radiation dose to be delivered to the patient, determine a homogeneous reference dose, calculate a complementary radiation dose by determining a difference between the homogeneous reference dose and the heterogeneous radiation dose, generate a treatment plan for the patient, the treatment plan including an optimized radiation dose to be delivered to the patient, combine the complementary radiation dose and the optimized radiation dose, evaluate the combined radiation dose with respect to the homogeneous reference dose, and display the combined radiation dose.

### RELATED APPLICATIONS

This application claims priority to U.S. Provisional Patent Application No. 61/157,062, filed on Mar. 3, 2009, the entire contents of which are incorporated herein by reference.

### BACKGROUND

Intensity modulation radiation therapy (“IMRT”) involves changing the size, shape, and intensity of a radiation beam to conform to the size, shape, and location of a tumor. IMRT is usually an automated process that is designed to deliver conformal radiation distributions to the tumor using a multi-leaf collimator programmed to modulate the dose as the MLC changes position.

IMRT involves the development of an optimized treatment plan, which generates the appropriate pattern, position, and intensity of the radiation beam based on the physician's dose prescription for how much radiation the tumor should receive, as well as acceptable levels for surrounding structures. The physician uses contours on one or more images to identify the target (e.g., tumor) and/or any regions at risk to define one or more boundaries or volumes and the amount of dose each volume should receive. The total prescribed dose of radiation in the treatment plan is divided into equal fraction sizes that are delivered to the patient at discrete times over the course of treatment (e.g., over a period of weeks rather than in a single session). The purpose of fractionation is to increase normal tissue sparing while simultaneously maintaining the same level of tumor cell kill, thereby increasing the therapeutic ratio. Fractionation therapy usually results in a better therapeutic ratio than single session therapy because it spares more normal tissue through repair of sub-lethal damage between dose fractions and re-population of cells.

During the course of treatment, it may become evident that a tumor includes a region of radiation resistance and/or a region of radiation sensitivity. Within the tumor, different levels of radiation dose may be necessary to boost radiation to the region of radiation resistance and/or to reduce radiation to the region of radiation sensitivity.

Adaptive radiation therapy (“ART”) is a technique used to modify the treatment plan between (or within) treatment fractions, based on feedback received from the radiation delivery device. The treatment plan can be modified to adjust or deviate from the prescribed dose, the amount of radiation to be delivered to these heterogeneous regions within the tumor.

### SUMMARY

Dose painting is a tool that allows medical personnel to specify different levels of radiation dose to be delivered to the patient. Dose painting provides for a deviation (i.e., a heterogeneous dose prescription) from a prescribed homogeneous dose to be delivered to the patient. A heterogeneous dose prescription is desired in many situations including re-optimization in ART to fix hot/cold spots from previous deliveries and dose boosting based on theragnostic imaging.

A process for “fixing” the hot/cold spots has been developed with minimal modification to the current treatment planning workflow. Currently, optimization of a treatment planning workflow is driven by a dose volume histogram (“DVH”)-based objective function, and the user relies on DVHs to evaluate the treatment plan quality. In one embodiment of the present invention, the user interface of the treatment planning software remains substantially the same whether evaluating a treatment plan including a homogeneous dose or a heterogeneous dose. If an embodiment of the invention were not implemented, the user interface would look substantially different when evaluating a treatment plan including a homogeneous dose or a heterogeneous dose. The difference in look is largely attributed to the difference in the appearance of the dose information and that the user would find it much less intuitive and more difficult to evaluate how good the treatment plan is because of the difference in appearance.

In one embodiment of the present invention, a method of optimizing a heterogeneous dose to be delivered to the patient and to retain the substantially similar user interface used in current treatment planning software includes determining or calculating a “complementary-dose,” which is the difference between a “homogeneous reference prescription” and a “heterogeneous prescribed dose distribution.” A treatment plan is generated and during each optimization iteration of the treatment plan, a “calculated-dose” (or optimized dose) to be delivered to the patient is generated and the “complementary-dose” is added to the “calculated-dose” to obtain a combined dose to be delivered to the patient. The combined dose is output (in the form of a dose map and/or a DVH) to the user to evaluate the combined dose with respect to the homogeneous reference prescription. This output allows the user to evaluate the combined dose as a DVH-based objective function which output is similar to what a user would see if only evaluating a treatment plan including a homogeneous dose. The ideal DVH is still a vertical line through the reference point. The DVH constraints for tumors and regions at risk are employed as in regular optimization.

Dose painting also can be implemented during re-optimization of a treatment plan in ART to fix previous errors as identified in a prior dose. During the adaptive process, one or more heterogeneous radiation doses have been delivered to the patient, and the dose to be delivered in the next fraction attempts to deliver a radiation dose that gets back to the originally-prescribed homogeneous radiation dose. In this process, an original homogeneous radiation dose has been prescribed for the patient but the resulting delivery of radiation over one or more fractions has been heterogeneous, due to one or more factors such as changes in machine output, mechanical error, or changes in the patient's position or anatomy. A current fraction with a heterogeneous radiation dose is being prescribed to try and smooth out the overall treatment to best match the original prescribed homogeneous dose. A complementary radiation dose is calculated by determining a difference between the homogeneous radiation dose and the heterogeneous radiation dose. Then a treatment plan is generated for the patient that includes an optimized radiation dose, which gets added to the complementary radiation dose to generate a combined radiation dose. The user can then evaluate the combined radiation dose to the heterogeneous radiation dose on the display.

Phantom studies were used to evaluate the feasibility of dose painting in the current treatment planning workflow. Various discrete and continuous prescribed dose distributions were tested. Dose profiles and effective DVHs were used to evaluate the results. For boosting discrete regions, the results show that the inventive process is able to resolve boost regions as small as 1 cm in diameter. Concave and convex continuous prescribed dose distribution, with gradient up to 20%/cm, can be well achieved with the inventive process.

In one embodiment, the invention provides a radiation therapy treatment system for optimizing radiation dose to be delivered to a patient. The radiation therapy treatment system comprises a computer processor and a software program stored in a computer readable medium accessible by the computer processor. The software program is operable to receive a prescribed heterogeneous radiation dose to be delivered to the patient, determine a homogeneous reference dose, calculate a complementary radiation dose by determining a difference between the homogeneous reference dose and the heterogeneous radiation dose, generate a treatment plan for the patient, the treatment plan including an optimized radiation dose to be delivered to the patient, combine the complementary radiation dose and the optimized radiation dose, evaluate the combined radiation dose with respect to the homogeneous reference dose, and display the combined radiation dose.

In another embodiment, the invention provides a method of optimizing radiation dose to be delivered to a patient. The method comprises generating a prescribed heterogeneous radiation dose to be delivered to the patient, generating a homogeneous reference dose, calculating a complementary radiation dose by determining a difference between the homogeneous reference dose and the heterogeneous radiation dose, generating a treatment plan for the patient, the treatment plan including an optimized radiation dose to be delivered to the patient, combining the complementary radiation dose and the optimized radiation dose, evaluating the combined radiation dose with respect to the homogeneous reference dose, and displaying the combined radiation dose.

In yet another embodiment, the invention provides a radiation therapy treatment system for optimizing radiation dose to be delivered to a patient. The radiation therapy treatment system comprises a computer processor and a software program stored in a computer readable medium accessible by the computer processor. The software program is operable to receive a prescribed homogeneous radiation dose to be delivered to the patient, calculate a heterogeneous radiation dose that has been previously delivered to the patient as a complementary dose, generate a treatment plan for the patient for at least one remaining treatment fraction, the treatment plan including an optimized radiation dose to be delivered to the patient, combine the complementary radiation dose and the optimized radiation dose, evaluate the combined radiation dose with respect to the prescribed homogeneous radiation dose, and display the combined radiation dose.

In another embodiment, the invention provides a method of optimizing radiation dose to be delivered to a patient. The method comprises generating a prescribed homogeneous radiation dose to be delivered to the patient, calculating a heterogeneous radiation dose that has been previously delivered to the patient as a complementary dose, generating a treatment plan for the patient for at least one remaining treatment fraction, the treatment plan including an optimized radiation dose to be delivered to the patient, combining the complementary radiation dose and the optimized radiation dose, evaluating the combined radiation dose with respect to the prescribed homogeneous radiation dose, and displaying the combined radiation dose.

In a further embodiment, the invention provides a method of evaluating a radiation dose. The method comprises generating a prescribed heterogeneous radiation dose to be delivered to the patient, generating a homogeneous radiation dose, retrieving a previously generated treatment plan, the treatment plan based on the prescribed heterogeneous radiation dose, calculating a complementary radiation dose by determining a difference between the homogeneous radiation dose and the prescribed heterogeneous radiation dose, applying the complementary radiation dose to the heterogeneous radiation dose to obtain a combined radiation dose, comparing the combined radiation dose with respect to the homogeneous radiation dose, and displaying the combined radiation dose.

In yet another embodiment, the invention provide a method of generating a user interface. The method comprises generating a treatment plan based on a heterogeneous dose prescription, applying an algorithm to the heterogeneous dose prescription to alter the heterogeneous dose prescription, calculating a dose volume histogram for the altered heterogeneous dose prescription that is visually similar to a dose volume histogram for a homogeneous dose prescription, and displaying the dose volume histogram to the user so the user can compare the treatment plan based on the heterogeneous dose prescription to a treatment plan based on a homogeneous dose prescription.

Other aspects of the invention will become apparent by consideration of the detailed description and accompanying drawings.

### BRIEF DESCRIPTION OF THE DRAWINGS

FIG. 1 is a perspective view of a radiation therapy treatment system.

FIG. 2 is a perspective view of a multi-leaf collimator that can be used in the radiation therapy treatment system illustrated in FIG. 1.

FIG. 3 is a schematic illustration of the radiation therapy treatment system of FIG. 1.

FIG. 4 is a schematic diagram of a software program used in the radiation therapy treatment system.

FIG. 5 illustrates an example of a prescribed homogeneous dose for a spherical shaped tumor.

FIG. 6 illustrates an example of a prescribed heterogeneous dose for the spherical shaped tumor in FIG. 5.

FIG. 7 illustrates an example of a complementary dose for the spherical shaped tumor in FIG. 5.

FIG. 8 illustrates an example of an optimized dose for the spherical shaped tumor in FIG. 5.

FIG. 9 illustrates an example of a combined dose for the spherical shaped tumor in FIG. 5.

FIG. 10 illustrates a DVH of the combined dose of FIG. 9 using a method of an embodiment of the present invention.

FIG. 11 illustrates a DVH of the optimized heterogeneous dose of FIG. 6 that does not use a method of an embodiment of the present invention.

FIG. 12 illustrates another example of a prescribed heterogeneous dose for a spherical shaped tumor.

FIG. 13 illustrates another example of a complementary dose for the spherical shaped tumor in FIG. 5.

FIG. 14 illustrates another example of an optimized dose for the spherical shaped tumor in FIG. 5.

FIG. 15 illustrates another example of a combined dose for the spherical shaped tumor in FIG. 5.

FIG. 16 illustrates a DVH of the combined dose of FIG. 15 using a method of an embodiment of the present invention.

FIG. 17 illustrates a DVH of the optimized heterogeneous dose of FIG. 12 that does not use a method of an embodiment of the present invention.

FIG. 18 includes four images illustrating dose painting for a discrete region of dose boosting in the treatment planning process.

FIG. 19 includes four images illustrating dose painting for a continuous varying prescribed dose distribution.

FIG. 20 includes four images illustrating dose painting for a concave-shaped prescribed dose distribution.

FIG. 21 includes four images illustrating dose painting for a concave-shaped prescribed dose distribution as in FIG. 20 except that a 3 cm diameter OAR (object at risk or avoidance region) is on the left side of the tumor bed.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first,” “second,” and “third” are used herein for purposes of description and are not intended to indicate or imply relative importance or significance.

In addition, it should be understood that embodiments of the invention include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

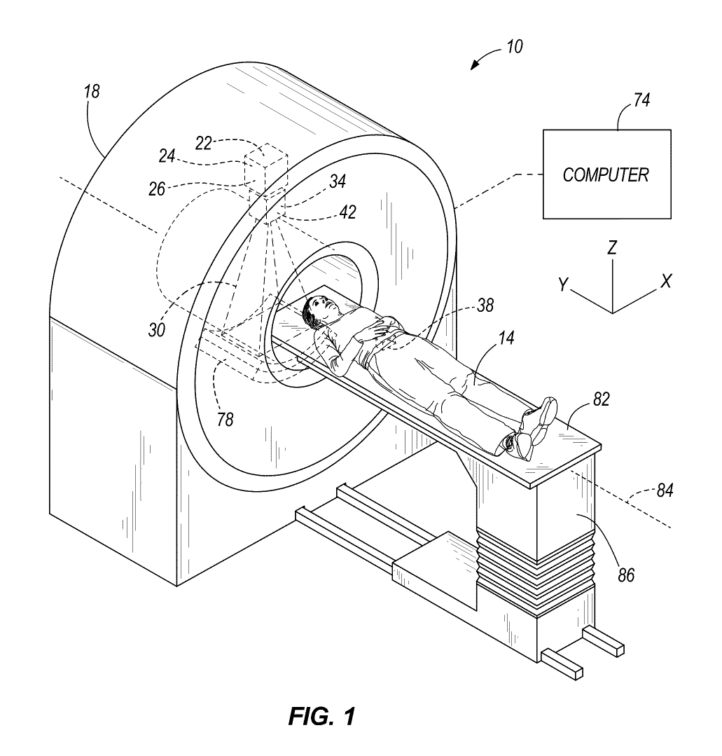

FIG. 1 illustrates a radiation therapy treatment system 10 that can provide radiation therapy to a patient 14. The radiation therapy treatment can include photon-based radiation therapy, brachytherapy, electron beam therapy, proton, neutron, or particle therapy, or other types of treatment therapy. The radiation therapy treatment system 10 includes a gantry 18. The gantry 18 can support a radiation module 22, which can include a radiation source 24 and a linear accelerator 26 (a.k.a. “a linac”) operable to generate a beam 30 of radiation. Though the gantry 18 shown in the drawings is a ring gantry, i.e., it extends through a full 360° arc to create a complete ring or circle, other types of mounting arrangements may also be employed. For example, a C-type, partial ring gantry, or robotic arm could be used. Any other framework capable of positioning the radiation module 22 at various rotational and/or axial positions relative to the patient 14 may also be employed. In addition, the radiation source 24 may travel in path that does not follow the shape of the gantry 18. For example, the radiation source 24 may travel in a non-circular path even though the illustrated gantry 18 is generally circular-shaped. The gantry 18 of the illustrated embodiment defines a gantry aperture 32 into which the patient 14 moves during treatment.

The radiation module 22 can also include a modulation device 34 operable to modify or modulate the radiation beam 30. The modulation device 34 provides the modulation of the radiation beam 30 and directs the radiation beam 30 toward the patient 14. Specifically, the radiation beam 30 is directed toward a portion 38 of the patient. Broadly speaking, a portion 38 may include the entire body, but is generally smaller than the entire body and can be defined by a two-dimensional area and/or a three-dimensional volume. A portion or area 38 desired to receive the radiation, which may be referred to as a target or target region, is an example of a region of interest. Another type of region of interest is a region at risk. If a portion 38 includes a region at risk, the radiation beam is preferably diverted from the region at risk. Such modulation is sometimes referred to as intensity modulated radiation therapy (“IMRT”).

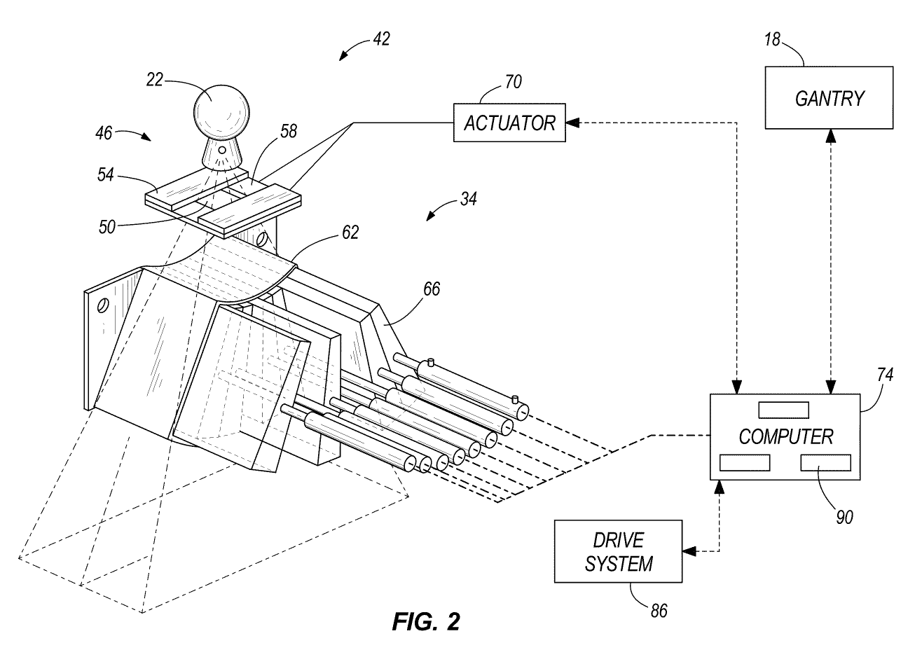

The modulation device 34 can include a collimation device 42 as illustrated in FIG. 2. The collimation device 42 includes a set of jaws 46 that define and adjust the size of an aperture 50 through which the radiation beam 30 may pass. The jaws 46 include an upper jaw 54 and a lower jaw 58. The upper jaw 54 and the lower jaw 58 are moveable to adjust the size of the aperture 50. The position of the jaws 46 regulates the shape of the beam 30 that is delivered to the patient 14.

In one embodiment, and illustrated in FIG. 2, the modulation device 34 can comprise a multi-leaf collimator 62 (a.k.a. “MLC”), which includes a plurality of interlaced leaves 66 operable to move from position to position, to provide intensity modulation. It is also noted that the leaves 66 can be moved to a position anywhere between a minimally and maximally-open position. The plurality of interlaced leaves 66 modulate the strength, size, and shape of the radiation beam 30 before the radiation beam 30 reaches the portion 38 on the patient 14. Each of the leaves 66 is independently controlled by an actuator 70, such as a motor or an air valve so that the leaf 66 can open and close quickly to permit or block the passage of radiation. The actuators 70 can be controlled by a computer 74 and/or controller.

The radiation therapy treatment system 10 can also include a detector 78, e.g., a kilovoltage or a megavoltage detector, operable to receive the radiation beam 30, as illustrated in FIG. 1. The linear accelerator 26 and the detector 78 can also operate as a computed tomography (CT) system to generate CT images of the patient 14. The linear accelerator 26 emits the radiation beam 30 toward the portion 38 in the patient 14. The portion 38 absorbs some of the radiation. The detector 78 detects or measures the amount of radiation absorbed by the portion 38. The detector 78 collects the absorption data from different angles as the linear accelerator 26 rotates around and emits radiation toward the patient 14. The collected absorption data is transmitted to the computer 74 to process the absorption data and to generate images of the patient's body tissues and organs. The images can also illustrate bone, soft tissues, and blood vessels. The system 10 can also include a patient support device, shown as a couch 82, operable to support at least a portion of the patient 14 during treatment. While the illustrated couch 82 is designed to support the entire body of the patient 14, in other embodiments of the invention the patient support need not support the entire body, but rather can be designed to support only a portion of the patient 14 during treatment. The couch 82 moves into and out of the field of radiation along an axis 84 (i.e., Y axis). The couch 82 is also capable of moving along the X and Z axes as illustrated in FIG. 1.

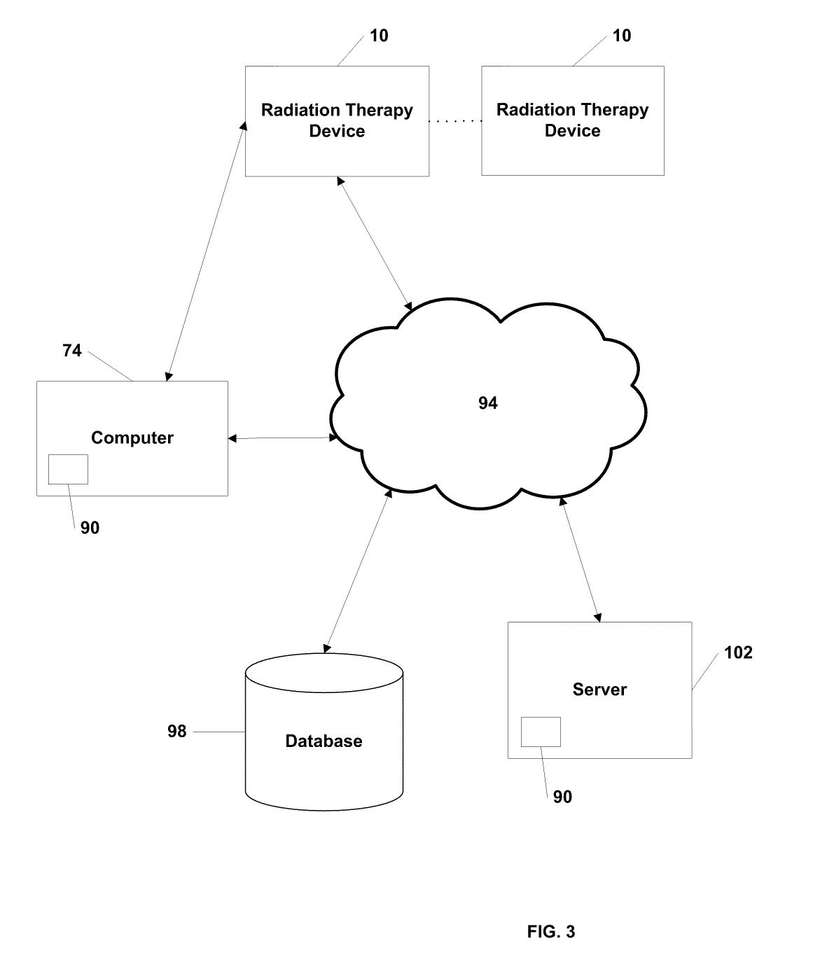

The computer 74, illustrated in FIGS. 2 and 3, includes an operating system for running various software programs and/or a communications application. In particular, the computer 74 can include a software program(s) 90 that operates to communicate with the radiation therapy treatment system 10. The computer 74 can include any suitable input/output device adapted to be accessed by medical personnel. The computer 74 can include typical hardware such as a processor, I/O interfaces, and storage devices or memory. The computer 74 can also include input devices such as a keyboard and a mouse. The computer 74 can further include standard output devices, such as a monitor. In addition, the computer 74 can include peripherals, such as a printer and a scanner.

The computer 74 can be networked with other computers 74 and radiation therapy treatment systems 10. The other computers 74 may include additional and/or different computer programs and software and are not required to be identical to the computer 74, described herein. The computers 74 and radiation therapy treatment system 10 can communicate with a network 94. The computers 74 and radiation therapy treatment systems 10 can also communicate with a database(s) 98 and a server(s) 102. It is noted that the software program(s) 90 could also reside on the server(s) 102.

The network 94 can be built according to any networking technology or topology or combinations of technologies and topologies and can include multiple sub-networks. Connections between the computers and systems shown in FIG. 3 can be made through local area networks (“LANs”), wide area networks (“WANs”), public switched telephone networks (“PSTNs”), wireless networks, Intranets, the Internet, or any other suitable networks. In a hospital or medical care facility, communication between the computers and systems shown in FIG. 3 can be made through the Health Level Seven (“HL7”) protocol or other protocols with any version and/or other required protocol. HL7 is a standard protocol which specifies the implementation of interfaces between two computer applications (sender and receiver) from different vendors for electronic data exchange in health care environments. HL7 can allow health care institutions to exchange key sets of data from different application systems. Specifically, HL7 can define the data to be exchanged, the timing of the interchange, and the communication of errors to the application. The formats are generally generic in nature and can be configured to meet the needs of the applications involved.

Communication between the computers and systems shown in FIG. 3 can also occur through the Digital Imaging and Communications in Medicine (DICOM) protocol with any version and/or other required protocol. DICOM is an international communications standard developed by NEMA that defines the format used to transfer medical image-related data between different pieces of medical equipment. DICOM RT refers to the standards that are specific to radiation therapy data.

The two-way arrows in FIG. 3 generally represent two-way communication and information transfer between the network 94 and any one of the computers 74 and the systems 10 shown in FIG. 3. However, for some medical and computerized equipment, only one-way communication and information transfer may be necessary.

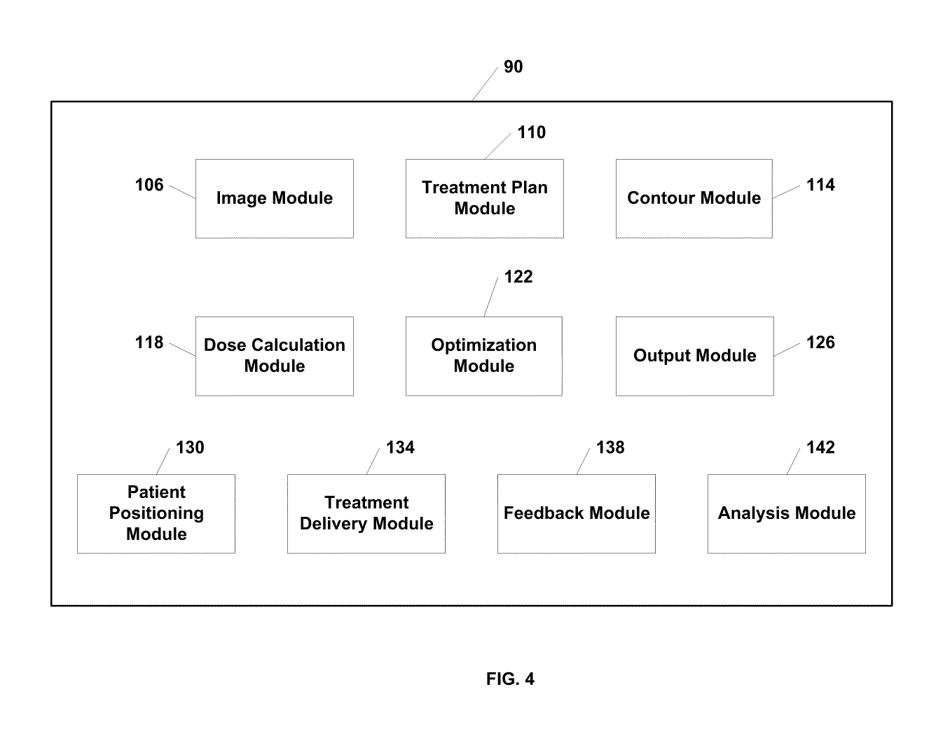

The software program 90 (illustrated in block diagram form in FIG. 4) includes a plurality of modules that communicate with one another to perform functions of the radiation therapy treatment process. The software program 90 can transmit instructions to or otherwise communicate with various components of the radiation therapy treatment system 10 and to components and/or systems external to the radiation therapy treatment system 10.

The software program 90 includes an image module 106 operable to acquire images of at least a portion of the patient 14. The image module 106 can instruct the on-board image device, such as a CT imaging device to acquire images of the patient 14 before treatment commences, during treatment, and after treatment according to desired protocols. In one aspect, the image module 106 acquires an image of the patient 14 while the patient 14 is substantially in a treatment position. Other off-line imaging devices or systems may be used to acquire pre-treatment images of the patient 14, such as non-quantitative CT, MRI, PET, SPECT, ultrasound, transmission imaging, fluoroscopy, RF-based localization, and the like. The acquired images can be used for registration of the patient 14 and/or to determine or predict a radiation dose to be delivered to the patient 14. The acquired images also can be used to generate a deformation map to identify the differences between one or more of the planning images and one or more of the pre-treatment, during-treatment, or after-treatment images. The acquired images also can be used to determine a radiation dose that the patient 14 received during the prior treatments. The image module 106 also is operable to acquire images of at least a portion of the patient 14 while the patient is receiving treatment to determine a radiation dose that the patient 14 is receiving in real-time.

The software program 90 includes a treatment plan module 110 operable to generate a treatment plan for the patient 14 based on data input to the system 10 by medical personnel. The data includes one or more images (e.g., planning images and/or pre-treatment images) of at least a portion of the patient 14. These images may be received from the image module 106 or other imaging acquisition device. The data also includes one or more contours received from or generated by a contour module 114. During the treatment planning process, medical personnel utilize one or more of the images to generate one or more contours on the one or more images to identify one or more treatment regions or avoidance regions of the portion 38. The contour process includes using geometric shapes, including three-dimensional shapes to define the boundaries of the treatment region of the portion 38 that will receive radiation and/or the avoidance region of the portion 38 that will receive minimal or no radiation. The medical personnel can use a plurality of predefined geometric shapes to define the treatment region(s) and/or the avoidance region(s). The plurality of shapes can be used in a piecewise fashion to define irregular boundaries. The medical personnel can identify the amount of radiation dose for the treatment region(s) and the avoidance region(s).

It is noted that the contours can be generated by the user in a manual process (i.e., the user can draw contours by freehand), can be generated automatically or semi-automatically (i.e., the software program 90 can automatically recognize the treatment region(s) and/or the avoidance region(s) to draw the contour), and/or can be generated in a deformation process. The user also can manually edit automatically generated contours.

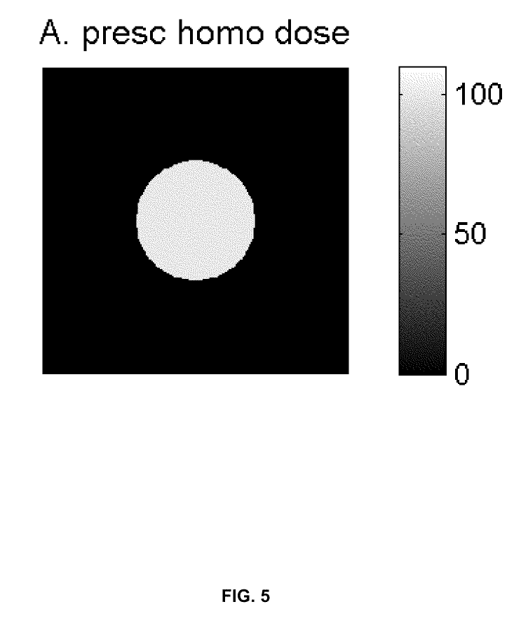

A physician or other medical personnel, during the treatment planning phase, utilize a dose calculation module 118 to provide a prescribed radiation dose amount and its distribution on the treatment region and/or the avoidance region of the portion 38. The dose calculation module 118 can determine the dose for a homogeneous dose delivery and for a heterogeneous dose delivery. Generally, the originally-prescribed radiation dose for the tumor is a homogeneous dose. FIG. 5 illustrates an example of a prescribed homogeneous dose. In this illustration, a homogeneous dose prescription of 100 Gy is shown for spherical shaped tumor.

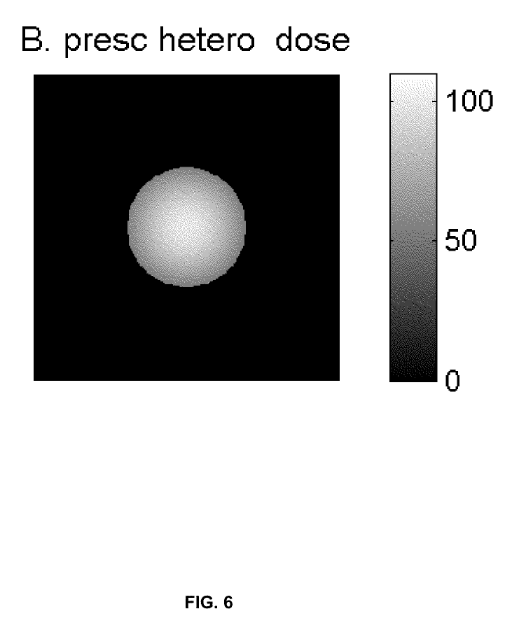

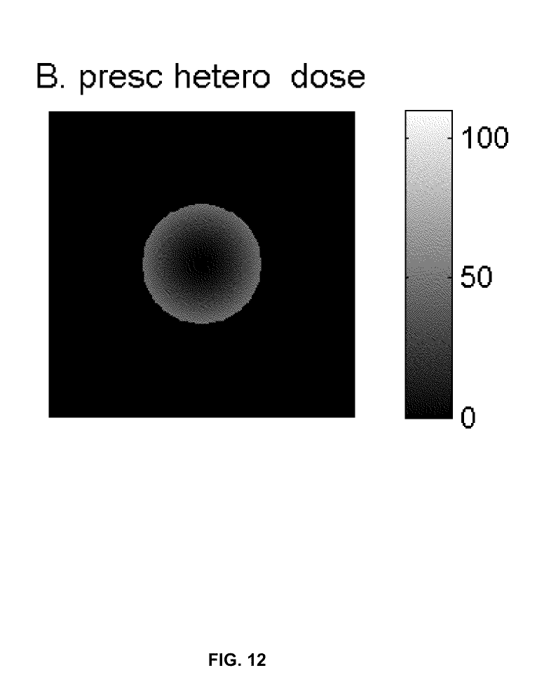

The dose calculation module 118 can separate the originally-prescribed radiation dose (e.g., homogeneous dose) into a plurality of fractions or treatments and determine the amount of radiation dose to be delivered to the patient during each fraction or treatment. Any one of the fractions of the originally-prescribed radiation dose can be modified to incorporate changes in the patient and changes in the system. During preparation for delivery of each fraction, medical personnel can modify the prescribed homogeneous dose to prescribe a heterogeneous dose for that fraction. FIGS. 6 and 12 illustrate examples of a prescribed heterogeneous dose. In this illustration, a heterogeneous dose prescription (two areas of the tumor to receive a different amount of dose) is shown for a spherical shaped tumor. The prescribed radiation dose (whether a homogeneous dose or a heterogeneous dose) can be based on the one or more contours drawn around the portion 38 that define the boundary or margin around the portion 38 and more specifically, the treatment region(s) and/or the avoidance region(s). Multiple portions 38 may be present and included in the same treatment plan.

The prescribed radiation dose can be viewed in a dose distribution, which illustrates an amount and location of the portion 38 that is going to receive the dose. The dose calculation module 118 can generate a two-dimensional plot called a dose volume histogram (“DVH”), which is a common method of analyzing the dose distribution, which is typically illustrated as three-dimensional volumes. The dose calculation module 118 can generate the DVH and display it on the screen/monitor for viewing by medical personnel. A DVH can include a plurality of subsets, which can include the dose volume curve and an area above and below the curve. This type of plot helps to provide an understanding of the range of doses provided to each portion 38 (which may include a region at risk). This can be useful during the treatment planning process for determining which structures may receive too much or too little dose and modifying the treatment plan accordingly. The treatment planning process can also use DVHs in a converse manner, which is to allow the user to view the DVH on the display/monitor and to select a region of the DVH curve to identify the portions of the 3D image or dose volumes that are receiving doses in a specified range. This method can assist in the treatment planning process since it can help the user better understand which regions are the most difficult to dose correctly.

The dose calculation module 118 also can recalculate a radiation dose to be delivered (e.g., in one or more fractions) to the patient 14 based on previous delivery information. The dose calculation module 118 can determine the effect that the location and/or movement of the patient had on the delivery of the prescribed radiation dose. The dose calculation module 118 can calculate an amount of radiation dose previously delivered to the patient 14. When reviewing or evaluating a treatment plan after a given delivery, medical personnel can review the dose amount delivered to the patient 14 and its effects in order to determine whether changes (e.g., a heterogeneous dose delivery for one or more fractions) need to be made to the treatment plan or a different plan(s) should be considered for future delivery of treatment.

When calculating the dose received by the patient, the dose calculation module 118 is operable to receive patient data (real-time and historic), patient images (e.g., planning images, pre-treatment images, and/or post-treatment images), patient position data, anatomical position data, and system or machine data. This data can be received from any one of the modules in the software program or directly from the system or machine. The dose calculation module 118 can provide information to the medical personnel related to the biological effect that the radiation dose has on the patient 14. The dose calculation module 118 can determine the biological effects of radiation on tissues, tumors, and organs based on the amount of radiation dose that the patient 14 has received and/or on the patient's registration. Based on the biological effects, the medical personnel can adjust the patient 14, the system settings, or make other adjustments in the treatment plan. The biological information can be incorporated in the patient registration process to identify a preferred position for the patient 14 that results in a delivered dose with a preferred biological effect.

The dose calculation module 118 can utilize data related to the radiation dose actually delivered and the biological effects of the radiation dose delivered and apply a biological model that relates the clinical radiation dose to the patient effect. The net radiation dose delivered (accumulation of radiation dose using deformation techniques) can be used to estimate the biological effect that would result from continuing the treatment, and likewise, possible alternatives for adapting the treatment can be evaluated for a preferred biological effect. The resulting fractionation schedule, dose distribution, and treatment plans can be modified and/or updated to reflect this culmination of information.

The software program 90 can include a treatment plan optimization module 122 operable to optimize or re-optimize the treatment plan generated by the treatment plan module 110. In particular, the optimization module 122 generates the commands or instructions for the radiation therapy treatment system 10 necessary to optimally deliver the treatment plan. The optimization module 122 is operable to determine and select between various parameters of operation of the radiation therapy treatment system 10 based on the type of treatment the patient 14 is going to receive and/or the mode of operation of the radiation therapy treatment system 10. Some of the parameters include, but are not limited to, position of the leaves 66, gantry angles and angular speed, speed of the drive system 86, type of motion of the couch 82, size of the jaw aperture 50, couch range of motion, and radiation beam intensity.

The optimization module 122 can optimize or re-optimize the treatment plan prior to treatment (e.g., delivery of any one of the fractions), but the optimization module 122 also can optimize or re-optimize the treatment plan in substantially real-time (e.g., during treatment delivery of any one of the fractions) to better take into account a variety of factors, such as patient anatomical and physiological changes (e.g., respiration and other movement, etc.), and machine configuration changes (e.g., beam output factors, couch error, leaf error, etc.). Real-time modification of the beam intensity can account for these changes (e.g., re-optimize beamlets in real time).

The optimization process performed by the optimization module 122 can account for cumulative errors and to adjust the treatment plan for future radiation delivered to the patient. The optimization module 122 can update the motion-encoded cumulative dose and optimize the leaf open time right before the delivery of each projection.

The optimization module 122 can communicate with the dose calculation module 118 to optimize a heterogeneous dose that has been prescribed for the patient for one or more fractions. During the optimization process, the optimization module 122 receives data, such as dose distribution(s) and DVHs from the dose calculation module 118 related to the newly-prescribed heterogeneous dose (see FIG. 6 or FIG. 12) and a homogeneous reference dose (see FIG. 5). The homogeneous reference dose may come from a portion of the prescribed heterogeneous dose, from an alternate treatment plan, and/or be based on a maximum homogeneous dose that cold be prescribed for certain areas of the tumor.

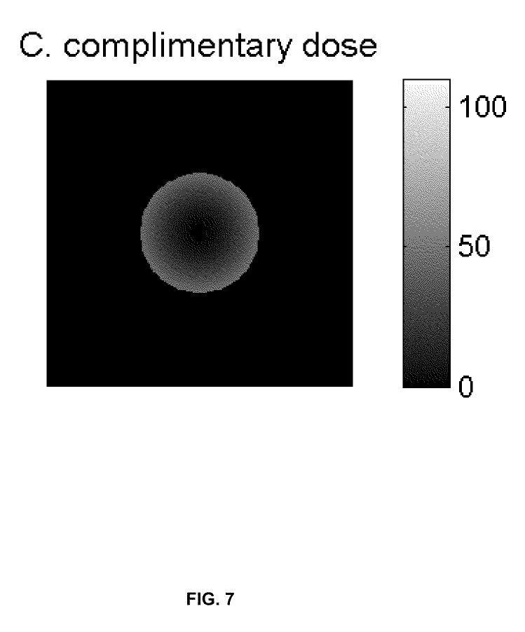

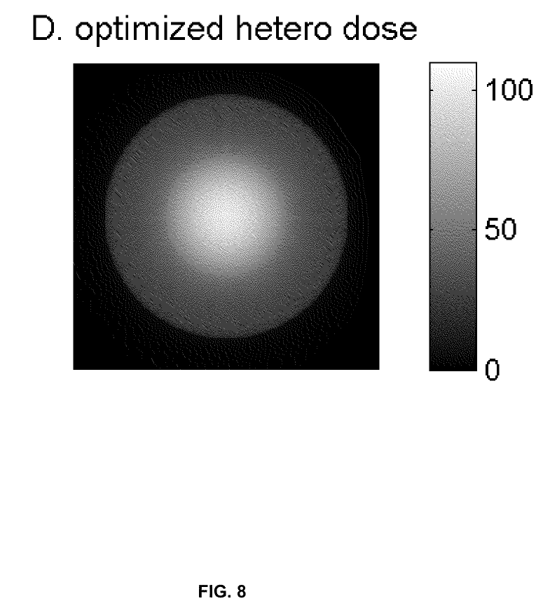

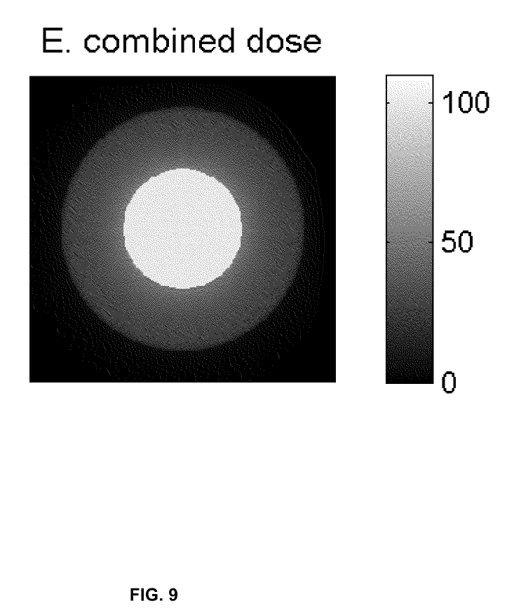

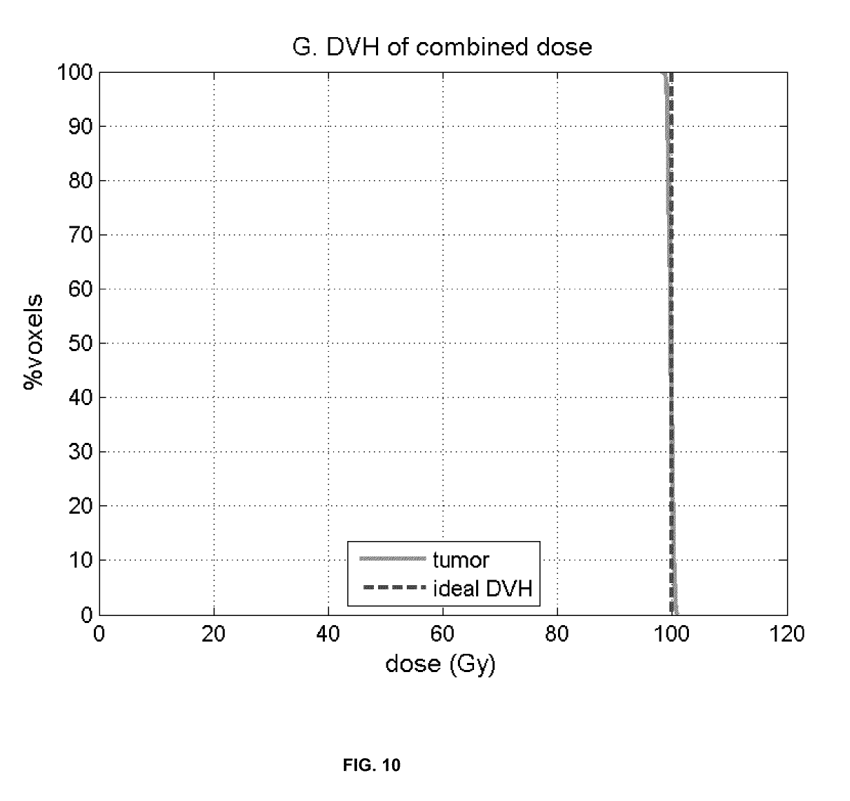

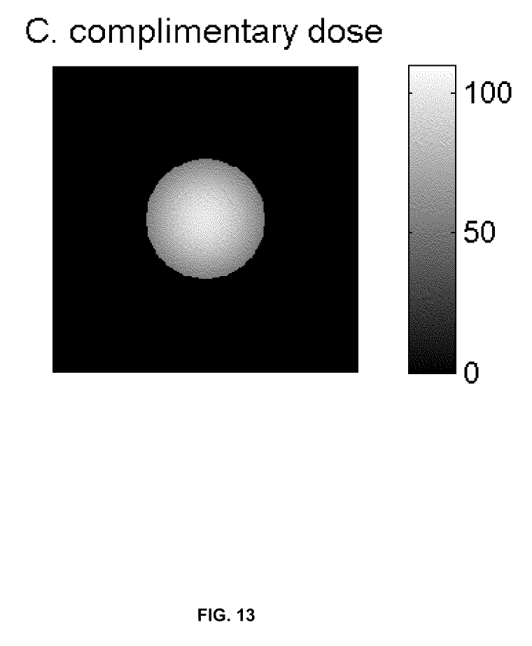

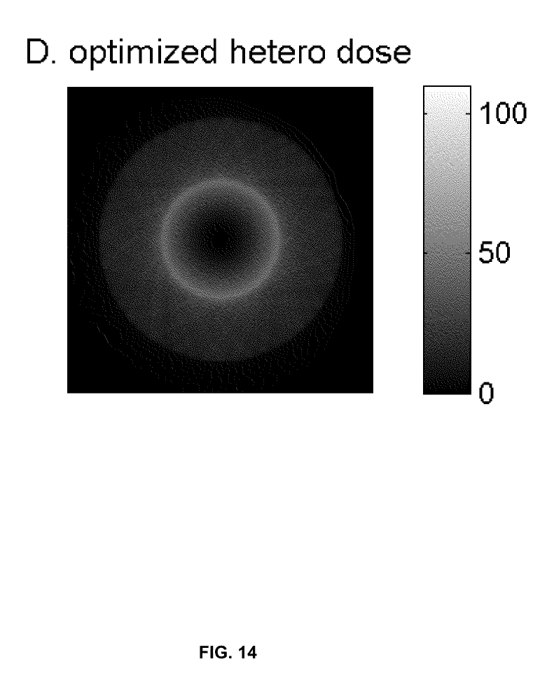

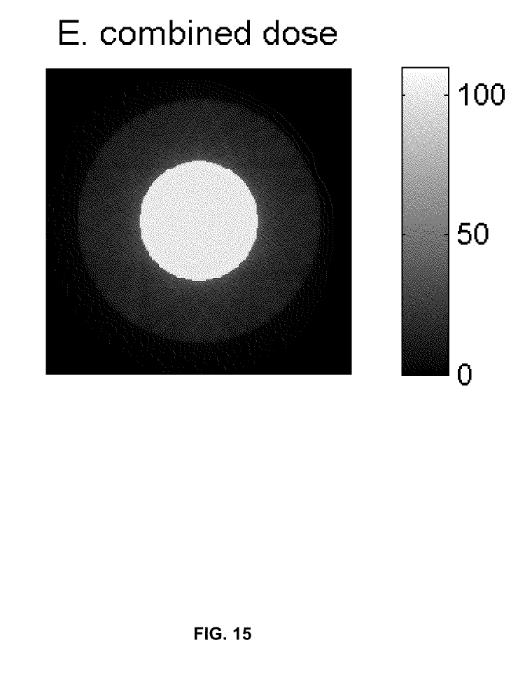

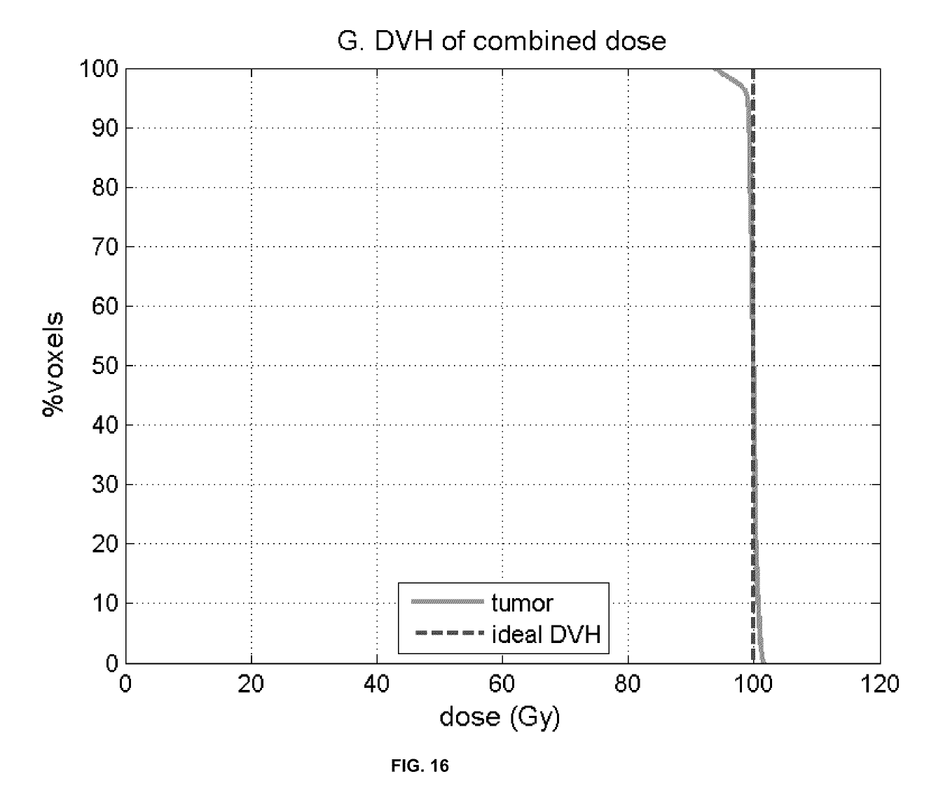

The optimization module 122 calculates a “complementary dose” which is the difference between the homogeneous reference dose and the heterogeneous prescribed distribution. FIGS. 7 and 13 illustrate examples of a complementary dose for a spherical shaped tumor. In the illustration of FIG. 7, the complementary dose is the subtraction of FIG. 6 from FIG. 5. In the illustration of FIG. 13, the complementary dose is the subtraction of FIG. 12 from FIG. 5. During the optimization process, an optimized treatment plan for the patient is generated that includes an optimized dose (e.g., heterogeneous dose). FIGS. 8 and 14 illustrate examples of an optimized dose for a spherical shaped tumor. The complementary dose is combined with the optimized dose to obtain a combined radiation dose to be delivered to the patient. FIGS. 9 and 15 illustrate examples of a combined dose for a spherical shaped tumor. In the illustration of FIG. 9, the combined dose is the combination of FIG. 7 and FIG. 8, which is similar to the homogeneous reference dose illustrated in FIG. 5. In the illustration of FIG. 15, the combined dose is the combination of FIG. 13 and FIG. 14, which is similar to the homogeneous reference dose illustrated in FIG. 5. The combined radiation dose can be evaluated by the medical personnel and compared to the homogeneous reference dose. The combined dose can be output to the user on the display for evaluation. The output can be in the form of a dose map and/or a DVH. A DVH illustrates the combined dose. FIGS. 10 and 16 illustrate a DVH of the combined dose of FIGS. 9 and 15, respectively. The ideal DVH for a heterogeneous dose is still a vertical line through the reference point as it would be for a homogeneous dose. FIGS. 10 and 16 illustrate this similarity to an ideal DVH (vertical straight line) to indicate that dose painting is well done. The DVH constraints for tumors and regions at risk are employed as in optimization of a treatment plan with a homogeneous dose.

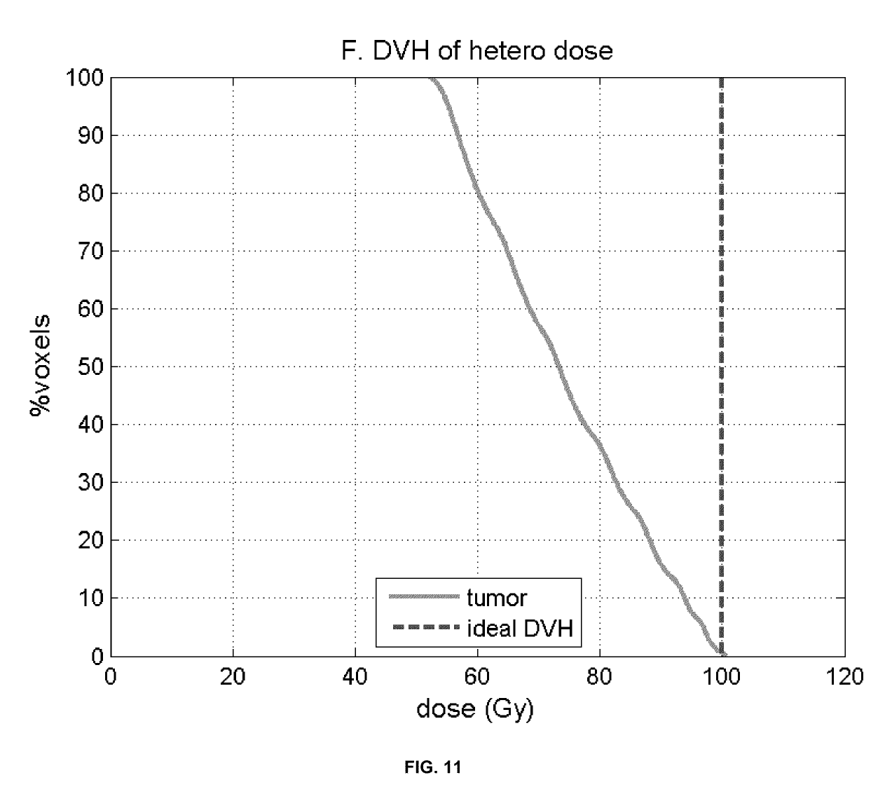

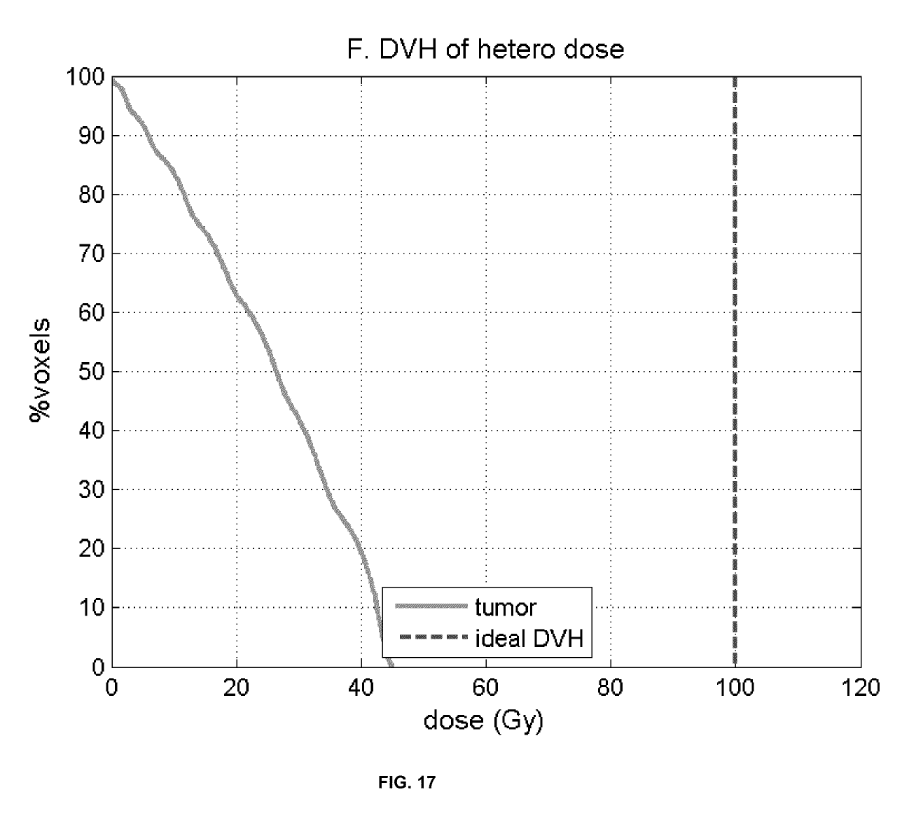

The software program performs this mathematical algorithm to generate and present a substantially similar user interface (see FIGS. 10 and 16) to the medical personnel to allow the medical personnel to evaluate one or more treatment plans that have been optimized for either a prescribed heterogeneous dose for one or more fractions or a prescribed homogeneous dose. If the mathematical algorithm was not performed, the user interface for the optimized heterogeneous dose would look substantially different than the user interface for the optimized homogeneous dose in that the dose output information (be it a DVH or otherwise) would look different. Due to those differences, the medical personnel may find it non-intuitive and difficult to evaluate how well the prescribed dose matches the physician's expectations. FIGS. 11 and 17 illustrate the output to the user if the mathematical algorithm was not performed. FIGS. 11 and 17 illustrate the DVH from a heterogeneous optimized dose vs. the ideal DVH for a homogeneous dose (vertical straight line). This DVH cannot tell how well the dose painting is done.

A similar method can be used for re-optimization in adaptive radiation therapy (“ART”) to fix previous errors as identified in a prior dose. In an adaptive process, the dose painting tool attempts to fix previous errors identified in a previously-delivered dose. During the adaptive process, one or more heterogeneous radiation doses have been delivered to the patient, and the dose to be delivered in the next fraction attempts to deliver a radiation dose that gets back to the originally-prescribed homogeneous radiation dose. In this process, an original homogeneous radiation dose has been prescribed for the patient but the resulting delivery of radiation over one or more fractions has been heterogeneous, due to one or more factors such as changes in machine output, mechanical error, or changes in the patient's position or anatomy, etc. A current fraction with a heterogeneous radiation dose is being prescribed to try and smooth out the overall treatment to best match the original prescribed homogeneous dose.

The optimization module 122 calculates a heterogeneous radiation dose that was previously delivered to the patient as a complementary dose. The complementary dose can be the difference between a homogeneous dose prescription and a heterogeneous prescribed distribution. Then a treatment plan is generated for the patient for at least one remaining treatment fraction. This treatment plan includes an optimized radiation dose, which gets added to the complementary radiation dose to generate a combined radiation dose. The user can then evaluate the combined radiation dose with respect to the prescribed homogeneous radiation dose. The combined dose can be output to the user on the display for evaluation. The output can be in the form of a dose map and/or a DVH. A DVH illustrates the combined dose. The ideal DVH for a heterogeneous dose is still a vertical line through the reference point.

The software program 90 also can include an output module 126 operable to generate or display data to the user via the user interface. The output module 126 can receive data from any one of the described modules, format the data as necessary for display and provide the instructions to the user interface to display the data. For example, the output module 126 can format and provide instructions to the user interface to display the combined dose in the form of a numerical value, a map, a histogram, or other suitable graphical illustration.

The software program 90 also includes a patient positioning module 130 operable to position and align the patient 14 with respect to the isocenter of the gantry 18 prior to or during the delivery of a particular treatment fraction. While the patient is on the couch 82 (e.g., substantially in a treatment position), the patient positioning module 130 can instruct the image module 106 to acquire an image of the patient 14. The patient positioning module 130 (or other module) can compare the current position of the patient 14 to the position of the patient in a reference image. The reference image can be a planning image, any pre-treatment image, or a combination of a planning image and a pre-treatment image. If the patient's position needs to be adjusted, the patient positioning module 130 can provide instructions to the drive system 86 to move the couch 82 or the patient 14 can be manually moved to the new position. In one construction, the patient positioning module 130 can receive data from lasers positioned in the treatment room to provide patient position data with respect to the isocenter of the gantry 18. Based on the data from the lasers, the patient positioning module 130 can provide instructions to the drive system 86, which moves the couch 82 to achieve proper alignment of the patient 14 with respect to the gantry 18. It is noted that devices and systems, other than lasers, can be used to provide data to the patient positioning module 130 to assist in the alignment process.

The patient positioning module 130 also is operable to detect and/or monitor patient motion during treatment. The patient positioning module 130 can communicate with and/or incorporate a motion detection system, such as x-ray, in-room CT, laser positioning devices, camera systems, spirometers, ultrasound, tensile measurements, chest bands, and the like. The patient motion can be irregular or unexpected, and does not need to follow a smooth or reproducible path.

The software program 90 also includes a treatment delivery module 134 operable to instruct the radiation therapy treatment system 10 to deliver the radiation fraction to the patient 14 according to the treatment plan. The treatment delivery module 134 can generate and transmit instructions to the gantry 18, the linear accelerator 26, the modulation device 34, and the drive system 86 to deliver radiation to the patient 14. The instructions coordinate the necessary movements of the gantry 18, the modulation device 34, and the drive system 86 to deliver the radiation beam 30 to the proper target in the proper amount as specified in the treatment plan.

The software program 90 also includes a feedback module 138 operable to receive data from the radiation therapy treatment system 10 during a patient treatment. The feedback module 138 can receive data from the radiation therapy treatment device and can include information related to patient transmission data, ion chamber data, MLC data, system temperatures, component speeds and/or positions, flow rates, etc. The feedback module 138 can also receive data related to the treatment parameters, amount of radiation dose the patient received, image data acquired during the treatment, and patient movement. In addition, the feedback module 138 can receive input data from a user and/or other sources. The feedback module 138 acquires and stores the data until needed for further processing.

The software program 90 also can include an analysis module 142 operable to analyze the data from the feedback module 138 to determine whether delivery of the treatment plan occurred as intended and to validate that the planned delivery is reasonable based on the newly-acquired data. The analysis module 142 can also determine, based on the received data and/or additional inputted data, whether a problem has occurred during delivery of the treatment plan. For example, the analysis module 142 can determine if the problem is related to an error of the radiation therapy treatment device 10, an anatomical error, such as patient movement, and/or a clinical error, such as a data input error.

The analysis module 142 can detect errors in the radiation therapy treatment device 10 related to the couch 82, the device output, the gantry 18, the multi-leaf collimator 62, the patient setup, and timing errors between the components of the radiation therapy treatment device 10. For example, the analysis module 142 can determine if a couch replacement was performed during planning, if fixation devices were properly used and accounted for during planning, if position and speed is correct during treatment.

The analysis module 142 can determine whether changes or variations occurred in the output parameters of the radiation therapy treatment device 10. With respect to the gantry 18, the analysis module 142 can determine if there are errors in the speed and positioning of the gantry 18. The analysis module 142 can receive data to determine if the multi-leaf collimator 62 is operating properly. For example, the analysis module 142 can determine if the leaves 66 move at the correct times, if any leaves 66 are stuck in place, if leaf timing is properly calibrated, and whether the leaf modulation pattern is correct for any given treatment plan. The analysis module 142 also can validate patient setup, orientation, and position for any given treatment plan. The analysis module 142 also can validate that the timing between the gantry 18, the couch 62, the linear accelerator 26, the leaves 66 are correct.

### Examples

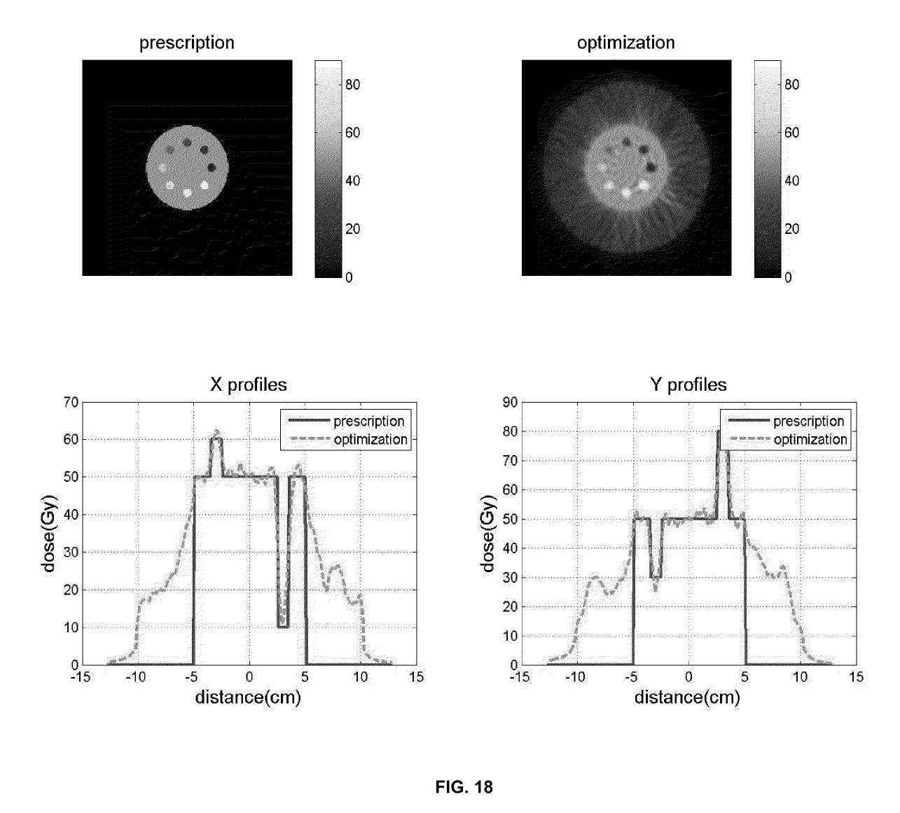

FIG. 18 illustrates discrete region dose boosting in the treatment planning process. The top left image graphically illustrates a prescribed dose distribution. The tumor bed is 10 cm in diameter with uniform prescription of 50 Gy. Inside the tumor bed, there are 8 round regions, each 1 cm in diameter, with prescribed dose (counter-clockwise) of 10, 20, 30, 40, 60, 70, 80, 90 Gy. The top right image illustrates an optimized dose distribution using dose painting. The bottom left and right images compare the prescribed (solid) and optimized (dashed) dose profiles. The bottom left image illustrates the profile along the X axis, while the bottom right image illustrates the profile along the Y axis. This figure indicates that boost regions of 1 cm in diameter can be well resolved via dose painting according to an embodiment of the present invention.

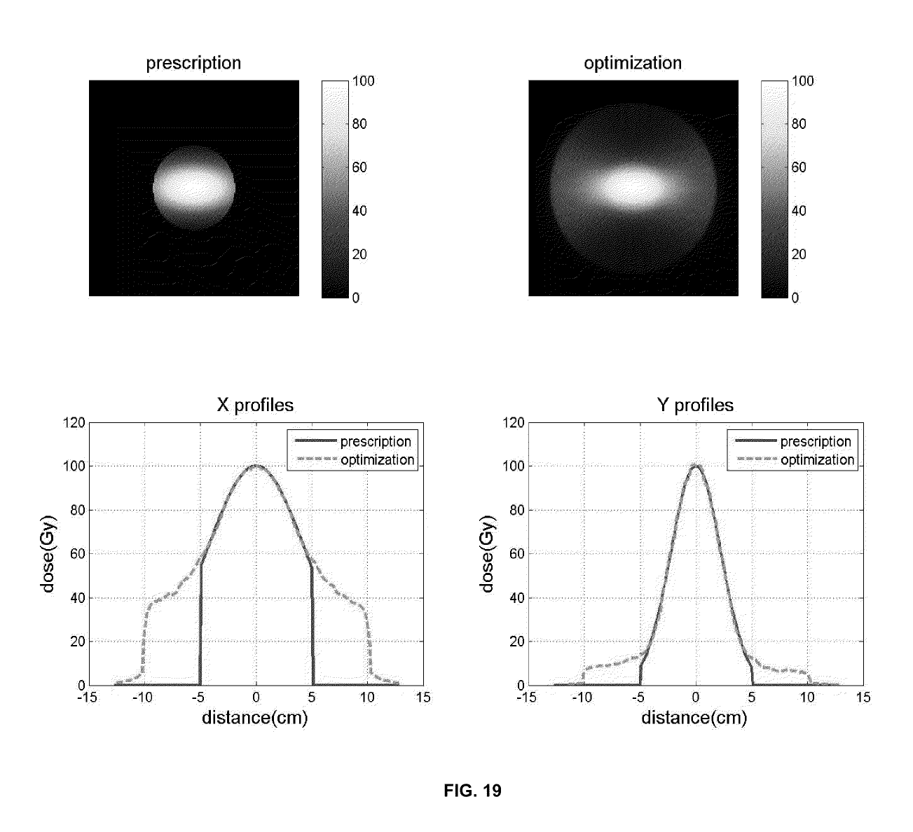

FIG. 19 illustrates dose painting for a continuous varying prescribed dose distribution. The top left image graphically illustrates a prescribed dose distribution. The top right image illustrates an optimized dose distribution using dose painting. The bottom left and right images compare the prescribed (solid) and optimized (dashed) dose profiles. The bottom left image illustrates the profile along the X axis, while the bottom right image illustrates the profile along the Y axis. This figure indicates that a prescribed dose gradient of 20%/cm can be well achieved via dose painting according to an embodiment of the present invention.

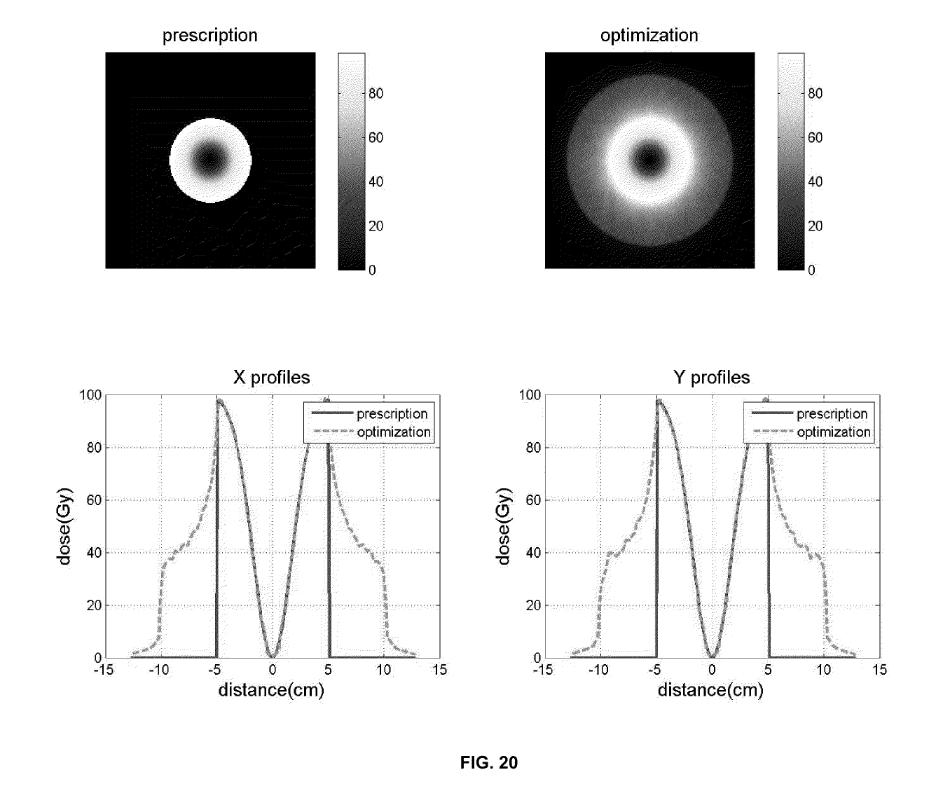

FIG. 20 illustrates dose painting for a concave shaped prescribed dose distribution. The top left image graphically illustrates a prescribed dose distribution. The top right image illustrates an optimized dose distribution using dose painting. The bottom left and right images compare the prescribed (solid) and optimized (dashed) dose profiles. The bottom left image illustrates the profile along the X axis, while the bottom right image illustrates the profile along the Y axis. This figure indicates that dose painting can handle concave-shaped tumors according to an embodiment of the present invention.

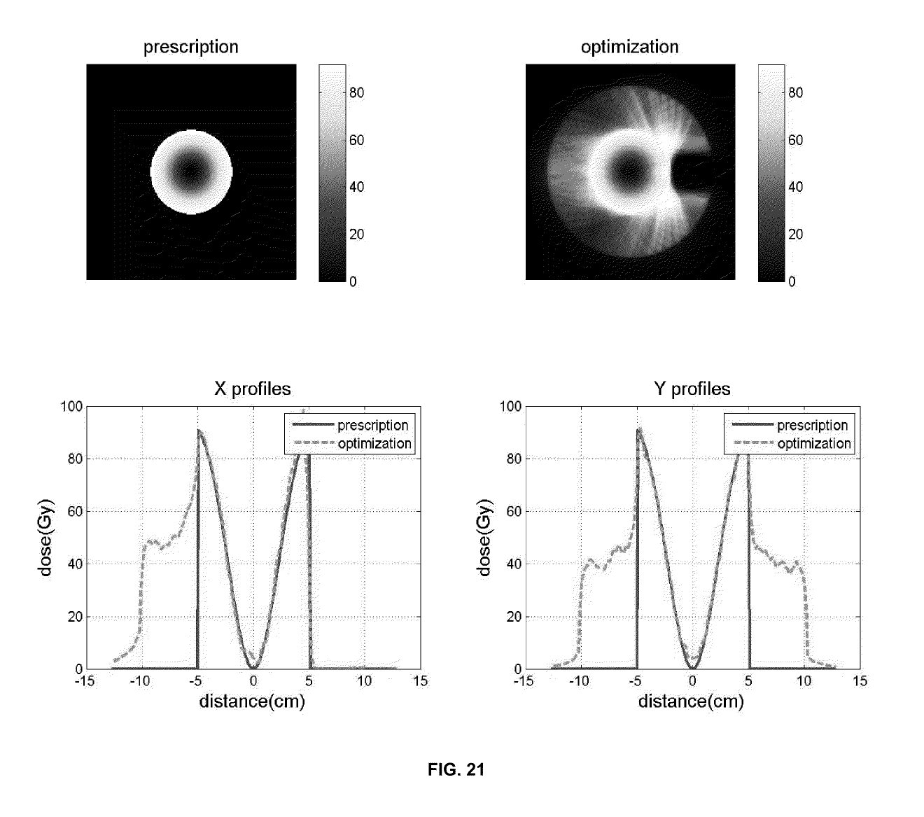

FIG. 21 illustrates dose painting for a concave-shaped prescribed dose distribution as in FIG. 20 except that a 3 cm diameter OAR (object at risk or avoidance region) is on the left side of the tumor bed. The top left image graphically illustrates a prescribed dose distribution. The top right image illustrates an optimized dose distribution using dose painting. The bottom left and right images compare the prescribed (solid) and optimized (dashed) dose profiles. The bottom left image illustrates the profile along the X axis, while the bottom right image illustrates the profile along the Y axis. The optimization result (top right) and the Y profile (bottom right) show that the OAR is avoided without sacrificing much of the tumor painting requirement via dose painting according to an embodiment of the present invention.

Various features and advantages of the invention are set forth in the following claims.

What is claimed is:

1. A radiation therapy treatment system for optimizing radiation dose to be delivered to a patient, the radiation therapy treatment system comprising:
a computer processor; and
a software program stored in a computer readable medium accessible by the computer processor, the software program being operable to
receive a prescribed heterogeneous radiation dose to be delivered to the patient,
determine a homogeneous reference dose,
calculate a complementary radiation dose by determining a difference between the homogeneous reference dose and the heterogeneous radiation dose,
generate a treatment plan for the patient, the treatment plan including an optimized radiation dose to be delivered to the patient,
combine the complementary radiation dose and the optimized radiation dose,
evaluate the combined radiation dose with respect to the homogeneous reference dose, and
display the combined radiation dose.

2. The radiation therapy treatment system of claim 1 wherein the combined radiation dose is displayed as a dose map.

3. The radiation therapy treatment system of claim 1 wherein the combined radiation dose is displayed as a dose volume histogram.

4. The radiation therapy treatment system of claim 1 wherein the prescribed heterogeneous radiation dose to be delivered to the patient is based at least in part on one or more MRI images.

5. The radiation therapy treatment system of claim 1 wherein the prescribed heterogeneous radiation dose to be delivered to the patient is based at least in part on one or more PET images.

6. The radiation therapy treatment system of claim 1 wherein the prescribed heterogeneous radiation dose to be delivered to the patient is based at least in part on one or more CT images.

7. The radiation therapy treatment system of claim 1 wherein the prescribed heterogeneous radiation dose to be delivered to the patient is based at least in part on a biological model.

8. The radiation therapy treatment system of claim 1 wherein the prescribed heterogeneous radiation dose to be delivered to the patient is based at least in part on dose already delivered to the patient.

9. The radiation therapy treatment system of claim 8 wherein the dose already delivered to the patient is evaluated using adaptive radiation therapy.

10. The radiation therapy treatment system of claim 1 wherein the prescribed heterogeneous radiation dose to be delivered to the patient is based at least in part on data from the system.

11. The radiation therapy treatment system of claim 1 wherein the optimized radiation dose to be delivered to the patient is an optimized heterogeneous radiation dose.

12. The radiation therapy treatment system of claim 1 further comprising generating a plurality of treatment plans for the patient.

13. The radiation therapy treatment system of claim 12 wherein each of the treatment plans includes a different optimized heterogeneous radiation dose to be delivered to the patient.

14. The radiation therapy treatment system of claim 1 further comprising receiving a plurality of prescribed heterogeneous doses for a plurality of targets.

15. A method of optimizing radiation dose to be delivered to a patient, the method comprising:
generating a prescribed heterogeneous radiation dose to be delivered to the patient;
generating a homogeneous reference dose;
calculating a complementary radiation dose by determining a difference between the homogeneous reference dose and the heterogeneous radiation dose;
generating a treatment plan for the patient, the treatment plan including an optimized radiation dose to be delivered to the patient;
combining the complementary radiation dose and the optimized radiation dose;
evaluating the combined radiation dose with respect to the homogeneous reference dose; and
displaying the combined radiation dose.

16. The method of claim 15 wherein displaying the combined radiation dose further comprises displaying the combined dose as a dose map.

17. The method of claim 15 wherein displaying the combined radiation dose further comprises displaying the combined dose as a dose volume histogram.

18. The method of claim 15 wherein the prescribed heterogeneous radiation dose to be delivered to the patient is based at least in part on one or more MRI images.

19. The method of claim 15 wherein the prescribed heterogeneous radiation dose to be delivered to the patient is based at least in part on one or more PET images.

20. The method of claim 15 wherein the prescribed heterogeneous radiation dose to be delivered to the patient is based at least in part on one or more CT images.

21. The method of claim 15 wherein the prescribed heterogeneous radiation dose to be delivered to the patient is based at least in part on a biological model.

22. The method of claim 15 wherein the prescribed heterogeneous radiation dose to be delivered to the patient is based at least in part on data from the system.

23. The method of claim 15 wherein the prescribed heterogeneous radiation dose to be delivered to the patient is based at least in part on dose already delivered to the patient.

24. The method of claim 23 wherein the dose already delivered to the patient is evaluated using adaptive radiation therapy.

25. The method of claim 15 wherein the optimized radiation dose to be delivered to the patient is an optimized heterogeneous radiation dose.

26. The method of claim 15 further comprising generating a plurality of treatment plans for the patient.

27. The method of claim 26 wherein each of the treatment plans includes a different optimized heterogeneous radiation dose to be delivered to the patient.

28. The method of claim 15 further comprising receiving a plurality of prescribed heterogeneous doses for a plurality of targets.

29. A radiation therapy treatment system for optimizing radiation dose to be delivered to a patient, the radiation therapy treatment system comprising:
a computer processor; and
a software program stored in a computer readable medium accessible by the computer processor, the software program being operable to
receive a prescribed homogeneous radiation dose to be delivered to the patient,
calculate a heterogeneous radiation dose that has been previously delivered to the patient as a complementary dose,
generate a treatment plan for the patient for at least one remaining treatment fraction, the treatment plan including an optimized radiation dose to be delivered to the patient,
combine the complementary radiation dose and the optimized radiation dose,
evaluate the combined radiation dose with respect to the prescribed homogeneous radiation dose, and
display the combined radiation dose.

30. The radiation therapy treatment system of claim 29 wherein the combined radiation dose is displayed as a dose map.

31. The radiation therapy treatment system of claim 29 wherein the combined radiation dose is displayed as a dose volume histogram.

32. The radiation therapy treatment system of claim 29 wherein the heterogeneous radiation dose is based at least in part on one or more MRI images.

33. The radiation therapy treatment system of claim 29 wherein the heterogeneous radiation dose is based at least in part on one or more PET images.

34. The radiation therapy treatment system of claim 29 wherein the prescribed heterogeneous radiation dose is based at least in part on one or more CT images.

35. The radiation therapy treatment system of claim 29 wherein the heterogeneous radiation dose is based at least in part on a biological model.

36. The radiation therapy treatment system of claim 29 wherein the heterogeneous radiation dose is based at least in part on data from the system.

37. The radiation therapy treatment system of claim 29 wherein the optimized radiation dose to be delivered to the patient is an optimized homogeneous radiation dose.

38. The radiation therapy treatment system of claim 29 further comprising generating a plurality of treatment plans for the patient.

39. The radiation therapy treatment system of claim 38 wherein each of the treatment plans includes a different optimized homogeneous radiation dose to be delivered to the patient.

40. The radiation therapy treatment system of claim 29 further comprising receiving a plurality of prescribed heterogeneous doses for a plurality of targets.

41. A method of optimizing radiation dose to be delivered to a patient, the method comprising:
generating a prescribed homogeneous radiation dose to be delivered to the patient;
calculating a heterogeneous radiation dose that has been previously delivered to the patient as a complementary dose;
generating a treatment plan for the patient for at least one remaining treatment fraction, the treatment plan including an optimized radiation dose to be delivered to the patient;
combining the complementary radiation dose and the optimized radiation dose;
evaluating the combined radiation dose with respect to the prescribed homogeneous radiation dose; and
displaying the combined radiation dose.

42. The method of claim 41 wherein displaying the combined radiation dose further comprises displaying the combined dose as a dose map.

43. The method of claim 41 wherein displaying the combined radiation dose further comprises displaying the combined dose as a dose volume histogram.

44. The method of claim 41 wherein the prescribed heterogeneous radiation dose is based at least in part on one or more MRI images.

45. The method of claim 41 wherein the prescribed heterogeneous radiation dose is based at least in part on one or more PET images.

46. The method of claim 41 wherein the prescribed heterogeneous radiation dose is based at least in part on one or more CT images.

47. The method of claim 41 wherein the prescribed heterogeneous radiation dose is based at least in part on a biological model.

48. The method of claim 41 wherein the prescribed heterogeneous radiation dose is based at least in part on data from the system.

49. The method of claim 41 wherein the optimized radiation dose to be delivered to the patient is an optimized homogeneous radiation dose.

50. The method of claim 41 further comprising generating a plurality of treatment plans for the patient.

51. The method of claim 50 wherein each of the treatment plans includes a different optimized homogeneous radiation dose to be delivered to the patient.

52. The method of claim 41 further comprising receiving a plurality of prescribed heterogeneous doses for a plurality of targets.

53. A method of evaluating a radiation dose, the method comprising:
generating a prescribed heterogeneous radiation dose to be delivered to the patient;
generating a homogeneous radiation dose;
retrieving a previously generated treatment plan, the treatment plan based on the prescribed heterogeneous radiation dose;
calculating a complementary radiation dose by determining a difference between the homogeneous radiation dose and the prescribed heterogeneous radiation dose;
applying the complementary radiation dose to the heterogeneous radiation dose to obtain a combined radiation dose;
comparing the combined radiation dose with respect to the homogeneous radiation dose; and
displaying the combined radiation dose.

54. A method of generating a user interface, the method comprising:
generating a treatment plan based on a heterogeneous dose prescription;
applying an algorithm to the heterogeneous dose prescription to alter the heterogeneous dose prescription;
calculating a dose volume histogram for the altered heterogeneous dose prescription that is visually similar to a dose volume histogram for a homogeneous dose prescription; and
displaying the dose volume histogram to the user so the user can compare the treatment plan based on the heterogeneous dose prescription to a treatment plan based on a homogeneous dose prescription.
